(19) United States
(12) Patent Application Publication
(10) Pub. No.: US 2018/0259514 A1
(43) Pub. Date: Sep. 13, 2018

(54) METHOD TO DETECT BLOOD CREATININE AND AN IMMUNOSUPPRESSIVE DRUG

(71) Applicant: TEVA PHARMACEUTICALS INTERNATIONAL GMBH, Jona (CH)

(72) Inventors: Leonard Hermen HOLTKAMP, Wounderberg (NL); Carla-Maria BRANDES, Fuerstenfeldbruck (DE); Ghita BOUKAMEL, Barr (FR); Ronen Meir TAMIR, Tenafly, NJ (US); Jeanne R. ANDRONOWITZ, Glen Mills, PA (US); Ron KAMIENCHICK, Kyriat Ono (IL); Jocelyn GILMOUR, Wayne, PA (US)

(21) Appl. No.: 15/764,336

(22) PCT Filed: Sep. 30, 2016
(86) PCT No.: PCT/EP2016/073495

§ 371 Date: Mar. 29, 2018

(30) Foreign Application Priority Data
Oct. 1, 2015 (GB) 1517386.7

(51) Int. Cl. G01N 33/558; G01N 33/70; G01N 33/94

(52) U.S. Cl. CPC G01N 33/558; G01N 33/70; G01N 2800/52; G01N 2800/245; G01N 2800/347; G01N 33/9493

(57) ABSTRACT

The invention provides a method for detecting or quantifying creatinine and an immunosuppressive drug selected from tacrolimus and ciclosporin in a blood sample, devices, including lateral flow assay devices, and kits, as well as methods that enables the reduction of nephrotoxicity and/or enables the maintenance of good renal function in an organ or tissue transplant patient who is undergoing treatment with an immunosuppressive drug selected from tacrolimus and ciclosporin, and methods for monitoring nephrotoxicity and/or kidney function in transplant patients.

### TECHNICAL FIELD

This invention concerns methods and devices for detecting or quantifying creatinine and an immunosuppressive drug selected from tacrolimus and ciclosporin in a blood sample. The methods and devices have applications in monitoring kidney function and blood levels of immunosuppressive drugs in transplant patients. In particular, the invention is useful in point of care devices.

### BACKGROUND OF THE INVENTION

Organ transplantation is the moving of an organ from one body to another or from a donor site to another location on the person's own body, to replace the recipient's damaged or absent organ. Organs and/or tissues that are transplanted within the same person's body are called autografts. Transplants that are recently performed between two subjects of the same species are called allografts. Allografts can either be from a living or cadaveric source.

Organs that can be transplanted include but are not limited to the heart, kidneys, liver, lungs, pancreas, intestine, and thymus. In addition, tissue such as bones, tendons, cornea, skin, heart valves, nerves and veins can also be transplanted.

Transplantation medicine is one of the most challenging and complex areas of modern medicine. Transplant rejection, during which the body has an immune response to the transplanted organ or tissue may lead to transplant failure. Because most human tissue and organ transplants are allografts, there is always a risk that the genetic differences between the organ or tissue and the recipient will mean that the recipient's immune system will identify the organ or tissue as foreign and attempt to destroy it, causing transplant rejection. The existence of a complex immune response system allows the body to distinguish between native and foreign material, and the proper functioning of the immune system is vital for the long term health, and although deficiencies in the immune response cause disease, in organ or tissue transplantation it may be appropriate to control the immune system. In treating or preventing allograft rejection, suppression of the immune system may be achieved using immunosuppressive drugs. The levels of these drugs, however, need to be carefully monitored to ensure that efficiency is maximised and toxicity is minimised. One of the most common toxic effects is a toxic effect on the kidneys. Kidney function can be monitored by methods including determining creatinine levels in blood.

Tacrolimus

Tacrolimus (also known as FK-506 or fujimycin, and sold under trade names including Advagraf, Prograf, Astagraf and Tacni), is an immunosuppressive drug. It can be used to reduce the activity of the patient's immune system in allograft organ transplant patients (e.g. liver, kidney or heart allograft recipients), to treat an ongoing organ or tissue rejection and/or to prevent organ or tissue rejection. It is a 23-membered macrolide lactone and is produced by Streptomyces tsukubaensis. It acts by reducing interleukin-2 (IL-2) production by T-cells, by preventing the dephosphorylation of NF-AT, which is required for the production of IL-2 and related cytokines.

Tacrolimus is normally prescribed to patients following allograft organ transplantation, to prevent rejection of the transplanted organ, as part of a post-transplant cocktail including steroids, mycophenolate, and IL-2 receptor inhibitors. Dosages are titrated to target blood levels. Typical starting doses for once-daily or twice-daily tacrolimus are 0.15-0.20 mg/kg body weight, and ongoing monitoring of blood levels is routinely carried out, in order to optimise dosing.

It is recommended that blood trough levels of tacrolimus should be monitored during the post-transplantation period, around twice weekly during the early post-transplant period and then periodically during maintenance therapy. It is also generally recommended that tacrolimus levels should also be monitored following dose adjustment, changes in the immunosuppressive regimen or following co-administration of substances which may alter tacrolimus whole blood concentrations.

Analysis of tacrolimus in whole blood can be performed by immunoassay or by liquid chromatography tandem mass spectrometry (LC-MS/MS). LC-MS/MS methods offer favourable analytical specificity and sensitivity but are expensive, can't be automated and need technically qualified staff to carry out and interpret the results. Laboratory based immunoassays offer around-the-clock results, operational flexibility and can be automated, but reagent costs are relatively high. A new electrochemiluminescence immunoassay (ECLIA) developed by Roche Diagnostics for use on cobas e immunoassay analyzers is available, but again requires specialised equipment [1].

Ciclosporin

Ciclosporin (also known as cyclosporin, cyclosporine, ciclosporin A, cyclosporine A, or cyclosporin A and sold under trade names including Equoral and Ciqorin, Sandimmune, Neoral, Cicloral, Gengraf and Deximune) is a further immunosuppressive drug that is widely used in allograft organ transplant patients to prevent rejection of the transplanted organ. It reduces the activity of the immune system by interfering with the activity and growth of T cells. Ciclosporin forms an intracellular complex with cyclophilline and this in turn inhibits Calcineurin. The role of Calcineurin is to help NFAT-P dephosphorylation into NFAT (nuclear factor of activated T-cells). This NFAT would go into the nucleus of the cell and would stimulate the DNA transcription for the formation of IL-2; thus by inhibiting calcineurin the immune activation is inhibited by ciclosporin. It is a cyclic peptide of 11 amino acids, produced by the fungus Beauveria nivea (Tolypocladium inflatum Gams).

It was originally used for preventing kidney and liver transplant rejection and is now also approved for the prevention of rejection of heart transplants. It is always used with adrenal corticosteroids and may also be administered with other compounds such as mycophenolate. In the EU the approved indications include organ transplantation (prevention of graft rejection following kidney, liver, heart, combined heart-lung, lung or pancreas transplants, treatment of transplant rejection in patients previously receiving other immunosuppressive agents), bone marrow transplantation (prevention of graft rejection following bone marrow transplantation and prophylaxis of graft-versus-host disease (GVHD), treatment of established graft-versus-host disease (GVHD), nephrotic syndrome (treatment of steroid dependent or steroid resistant nephrotic syndrome (associated with adverse prognostic features) due to minimal change glomerulonephritis, focal segmental glomerulosclerosis or membranous glomerulonephritis in both adults and children. It can also be used to maintain steroid-induced remission, allowing to suspend steroids, rheumatoid arthritis (indicated for the treatment of severe, active rheumatoid arthritis in patients in whom classical, disease modifying anti-rheumatic drugs (DMARD's) are inappropriate or ineffective), psoriasis (treatment of severe forms of psoriasis in patients in whom conventional therapy is inappropriate or ineffective), and atopic dermatitis (indicated in patients with severe atopic dermatitis in whom conventional therapy is ineffective).

Blood concentration monitoring of ciclosporin can be useful in patient management because ciclosporin has a narrow therapeutic range with frequent adverse effects. Dose is adjusted initially (up to two months post-transplant) to maintain concentrations generally between 150 and 400 ng/mL. Target trough concentrations vary according to the clinical protocol and depend on factors including the type of allograft, the risk of rejection, the nature and/or concentration of concomitant immunosuppressive drugs, and toxicity. After the first two postoperative months, the target range is generally lower, between 75 and 300 ng/mL.

For existing ciclosporin assays, whole blood is the preferred specimen for analysis. HPLC and HPLC-MS/MS methods are generally used. As well as these methods, monoclonal specific radioimmunoassays (mRIA-sp) exist, as do nonspecific assays which detect the parent compound molecule and various of its metabolites.

Creatinine

Creatinine is a by-product of muscle metabolism that is excreted unchanged by the kidneys. For this reason, the levels of creatinine in the blood are used as an important indicator of renal function. The kidneys are primarily responsible for the removal of creatinine from the blood and deficient filtration in the kidney causes creatinine blood levels to rise.

Measuring serum creatinine is one of the most commonly used indicators of renal function. Serum creatinine is commonly measured by alkaline picrate (Jaffe method), enzymatic, and high-performance liquid chromatography (HPLC) methods. These different methods of measuring serum creatinine are standardised to the isotope dilution mass spectrometry (IDMS).

IDMS is highly specific and offers the most accurate results for serum creatinine, but is available only in selected laboratories. Combining HPLC and IDMS also provides highly accurate results for serum creatinine, but it has limited availability. HPLC methods have better specificity than the conventional methods and are less prone to interference, especially if combined with sample deproteinisation. Electronic Point-of-care testings (POCT) such as StatSensor® Xpress™ Creatinine (Nova Biomedical) are also available in healthcare settings and are considered to be sufficiently accurate for clinical use [2].

Detecting Immunosuppressive Drug and Creatinine Together

Monitoring the blood levels of each of the immunosuppressive drugs mentioned above forms part of the overall process of monitoring patients (e.g. transplant patients) treated with these drugs, although other tests are also performed on a regular basis. As noted above, kidney function can be monitored by testing blood (e.g. serum) creatinine. Kidney function may be compromised in patients (e.g. transplant patients) who are taking tacrolimus or ciclosporin because tacrolimus and ciclosporin both have nephrotoxic potential. Indeed, in ciclosporin-treated patients, nephrotoxicity was noted in 25% of cases of renal transplantation, 38% of cases of cardiac transplantation, and 37% of cases of liver transplantation. Mild nephrotoxicity (generally noted 2 to 3 months after transplant) is furthermore often responsive to immunosuppressive drug dosage reduction. It has been recognised that one of the main challenges for transplantation medicine is achieving good long-term outcome for patients, and new methods to improve the way in which the detection of immunosuppressive drugs and creatinine is carried out may contribute to better outcomes. Because of the interplay between the blood level of immunosuppressive drugs and kidney nephrotoxicity, and the potential to influence kidney nephrotoxicity by modulating the blood level of these immunosuppressive drugs by modulating administration doses of the immunosuppressive drugs, monitoring kidney function in parallel with monitoring the blood level of immunosuppressive drugs is one way to achieve such improvements.

Monitoring kidney function is also a way of determining the health of a transplanted kidney. In kidney transplant patients therefore kidney function is monitored as a means to monitor the function of the graft.

At present, kidney function is assayed by testing creatinine concentrations in the blood or serum, or in urine, whilst blood samples would separately be analysed for immunosuppressive drug levels using one of more of the assays discussed above. New methods are needed for the regular, efficient, and accurate monitoring of immunosuppressive drug levels and renal function. These methods may be more convenient than the currently available methods and/or may facilitate patient compliance, improve patient prognosis, and improve quality of life. Also needed are devices for use in the disclosed methods.

### DISCLOSURE OF THE INVENTION

The invention concerns new methods for obtaining information to enhance the monitoring of patients who have been administered an immunosuppressive drug selected from tacrolimus and ciclosporin, e.g. post-transplant monitoring of allograft transplant patients, and involves a method of detecting or quantifying creatinine and an immunosuppressive drug selected from tacrolimus and ciclosporin in a blood sample. Detecting or quantifying creatinine and an immunosuppressive drug selected from tacrolimus and ciclosporin in a blood sample provides improvements compared to known methods of detecting or quantifying creatinine and detecting or quantifying an immunosuppressive drug selected from tacrolimus and ciclosporin in a blood sample. This assists in monitoring and modifying treatment regimens for these patients.

The improvements may take one of several forms.

Allowing Simultaneous Detection of Creatinine and the Immunosuppressive Drug in a Single Sample

Methods which allow the simultaneous detection of creatinine and the immunosuppressive drug in a single sample can reduce the amount of sample that is required, and reduce the number of assays that need to be carried out in order to obtain the required results.

Improved Speed of Obtaining the Results Compared to Existing Assays

Providing both patients and doctors with information that can inform treatment earlier would be advantageous. By using assays such as immunoassays to detect creatinine and immunosuppressive drugs, the results can be obtained more quickly than methods involving laboratory analysis. This has the potential to influence patient outcome positively.

Allowing the Assays to be Carried Out Using a Point of Care Device

Better patient compliance with regard to drug administration and more rapid intervention in the case of adverse events can be achieved if patients are monitored more intensely. This in turn leads to better outcomes, but repeated travelling to hospitals or clinics can be onerous. Therefore methods which avoid this need would be advantageous. The use of point of care devices, e.g. based on immunoassays e.g. including lateral flow assays, can therefore be of great potential.

According to a first aspect of the invention, the invention provides a method for detecting or quantifying creatinine and an immunosuppressive drug selected from tacrolimus and ciclosporin in a blood sample.

The invention also provides a device for carrying out the method of the invention, e.g. a lateral flow device suitable for carrying out the lateral flow assay, as described herein.

The invention also provides a kit for performing the methods described herein. Suitable kits comprise (i) devices and/or reagents sufficient for performing at least one of the described methods, and (ii) instructions for performing the methods.

The invention further provides a method that enables the reduction of nephrotoxicity and/or enables the maintenance of good kidney function in a patient (e.g. an organ or tissue transplant patient) who is undergoing treatment with an immunosuppressive drug selected from tacrolimus and ciclosporin at a first dose, the method comprising quantifying creatinine and the immunosuppressive drug in a blood sample from the patient, and if the level of creatinine is indicative of nephrotoxicity and/or reduced kidney function, determining a new dose of the immunosuppressive drug for the patient.

The method can also be defined as a method for determining nephrotoxicity and immunosuppressive drug levels in a patient (e.g. an organ or tissue transplant patient) who is undergoing treatment with an immunosuppressive drug selected from tacrolimus and ciclosporin, the method comprising quantifying creatinine and the immunosuppressive drug in a blood sample from the patient.

The method can also be defined as a method for determining renal function and immunosuppressive drug levels in a patient (e.g. an organ or tissue transplant patient) who is undergoing treatment with an immunosuppressive drug selected from tacrolimus and ciclosporin, the method comprising quantifying creatinine and the immunosuppressive drug in a blood sample from the patient.

The invention further provides a method for monitoring renal function and immunosuppressive drug levels in a patient (e.g. an organ or tissue transplant patient) who is undergoing treatment with an immunosuppressive drug selected from tacrolimus and ciclosporin, the method comprising quantifying creatinine and the immunosuppressive drug in a blood sample from the patient. The invention further provides a method for monitoring nephrotoxicity and immunosuppressive drug levels in a patient (e.g. an organ or tissue transplant patient) who is undergoing treatment with an immunosuppressive drug selected from tacrolimus and ciclosporin, the method comprising quantifying creatinine and the immunosuppressive drug in a blood sample from the patient.

The invention also provides the use of the methods, devices and kits of the invention in monitoring a a patient (e.g. an organ or tissue transplant patient) who is undergoing treatment with an immunosuppressive drug selected from tacrolimus and ciclosporin.

### DETAILED DESCRIPTION OF THE INVENTION

Immunoassays

The method of the invention is preferably an immunoassay. Immunoassays are particularly useful as they are both rapid and specific. In immunoassays, use is made of the specificity of antibodies to particular analyte(s) (in this case creatinine and an immunosuppressive drug selected from tacrolimus and ciclosporin) to detect the analyte(s). The presence or amount of analyte is generally determined using methods in which an antibody specific for each analyte is used and specific binding is detected. Specific binding of the antibody to the analyte can be detected directly or indirectly.

The immunoassay can be a sandwich assay or a competitive assay.

In an exemplary sandwich assay, detection of the analyte is based on the specific binding of the analyte to two antibodies, at least one of which is labeled to enable detection. The detection of the presence or amount of the sandwich (antibody-analyte-antibody) provides the detection of the presence or amount of the analyte. In general, one antibody captures the analyte and the other antibody enables the detection of the presence or amount of the analyte. The two antibodies bind to the same but may bind to different epitopes on the analyte.

In an exemplary competitive assay, detection of the analyte is based on the ability of the analyte to compete with a modified version thereof, for binding to an antibody. Each of the analyte and the modified version thereof can bind to the antibody, but not at the same time. In the absence of analyte the modified version of the analyte (which is present in a defined amount) binds to the antibody (also present in a predetermined amount). If any analyte is present, it competes with the modified version thereof, resulting in a reduction in binding of the modified version to the antibody. This reduction in binding provides the indication of the presence of the analyte. Low analyte concentrations in the sample tend to result in the modified analyte binding to the antibodies, while high analyte concentrations in the sample cause the labelled analyte to be displaced from the antibodies.

In competitive assays, the presence of the analyte in the sample is thus indicated by the absence of binding of the modified version of the analyte to the antibody. Competitive assays are preferred as they are particularly useful for low molecular weight molecules in view of the fact that it is not necessary for more than one antibody binding site to be present on the analyte.

The method of the invention thus may include the steps of carrying out an immunoassay as described above, such as a sandwich or competitive immunoassay. Such a method may involve the steps of contacting the sample with the necessary reagents as described above (e.g. a modified version of the analyte or one or more antibody e.g. to the analyte) and detecting or quantifying the analyte on the basis of the assay.

Antibodies

The term “antibody” as used herein includes antibody fragments that are capable of specifically binding an antigen or epitope. The term antibody therefore includes antigen-binding portions, i.e., “antigen binding sites,” (e.g., fragments, subsequences, complementarity determining regions (CDRs)) that retain capacity to bind antigen, including F(ab′)2 fragments ((110,000 daltons) two antigen-binding regions joined at the hinge through disulfides), Fab′ fragments ((55,000 daltons) fragments that are e.g. formed by the reduction of F(ab′)2 fragments and contain a free sulfhydryl group), Fab fragments ((50,000 daltons) monovalent fragments that are produced from IgG and IgM, consisting of the VH, CH1 and VL, CL regions, linked by an intramolecular disulfide bond), Fv fragments ((25,000 daltons) the smallest fragment produced from IgG and IgM that contains a complete antigen-binding site), “rIgG” ((75,000 daltons), the product of selectively reducing just the hinge-region disulfide bonds). Single chain antibodies are also included by reference in the term “antibody.”

Preferably, an antibody is selected that specifically binds the target (e.g. the analyte or modified version thereof). The term “specifically binds” is not intended to indicate that an antibody binds exclusively to the target. Rather, an antibody “specifically binds” if its affinity for its intended target is about 5-fold greater when compared to its affinity for a non-target molecule. Preferably the affinity of the antibody will be at least about 5-fold, preferably 10-fold, more preferably 25-fold, even more preferably 50-fold, and most preferably 100-fold or more, greater for a target molecule than its affinity for a non-target molecule. In preferred embodiments, specific binding between an antibody and an antigen means a binding affinity of at least 10⁻⁶M. Preferred antibodies bind with affinities of at least about 10⁻⁷M, 10⁻⁸M, 10⁻⁹M or 10⁻¹⁹M, and preferably between about 10⁻⁸M to about 10⁻¹⁹M. Antibody affinity measurement e.g. by Scatchard analysis is well known in the art (see, e.g. [3]), as are methods to generate and select appropriate antibodies.

Where a competitive immunoassay is used, the antibody preferably binds specifically to each of the analyte and the modified version thereof. More preferably it binds specifically to each of the analyte and the modified version thereof with about the same or with the same affinity.

Preferably antibodies are monoclonal antibodies.

Modified Analyte

The “modified analyte” which may be used in the competitive assays according to the invention will be modified compared to the analyte in a way which does not influence the binding of the antibody that is used. The modification may be e.g. to include a label for detection, as defined elsewhere herein, or to immobilise the analyte on a solid support.

Labels

Labels for use in immunoassays may include molecules that are themselves detectable (e.g., fluorescent or luminescent molecules, metals (e.g. gold), dyes, magnetic particles, radionuclides etc.) as well as molecules that may be detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.). Particularly preferred detectable labels are coloured or fluorescent labels. IR and NIR dyes can also be used (see e.g. [4]).

The labels may be attached directly to the relevant molecule (e.g. to the antibody or modified version of the analyte). In some cases, the labels are attached to or are in or may be present in the form of particles. Examples include coloured or fluorescent particles such as coloured or fluorescent latex particles or gold particles. Molecules may also be labelled indirectly (e.g. where a label is attached to a further molecule that binds to that molecule). Therefore an antibody could be labelled by attaching a label directly to it, or by attaching a label directly to a further antibody that binds to the first antibody. Such labeling techniques are well known in the art. A single label, or multiple labels can be used in each assay or device according to the invention.

Solid Phase

One or more of the components of the immunoassay, such as the antibodies in the immunoassay may be immobilized on a solid phase.

Immobilisation of the relevant molecules can be carried out by any suitable means and such means are well known in the art. The immobilised molecules remain available for binding to their binding partner.

In a competitive imunoassay, either the modified version of the analyte or an antibody to the analyte can be immobilised on a solid phase. If an antibody is immobilised, the modified version of the analyte may be labeled (directly or indirectly) to allow for the detection of binding.

If the modified version of the analyte is immobilised, the modification will in general be the immobilisation of the analyte. In such a case the antibody may be labelled (directly or indirectly) to allow for the detection of binding.

The term “solid phase” as used herein refers to a wide variety of materials including solids, semi-solids, gels, films, membranes, meshes, felts, composites, particles, papers and the like typically used by those of skill in the art to sequester molecules. The solid phase can be non-porous or porous. Preferred solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. Nitrocellulose membranes are commonly used in the context of lateral flow assays and are highly preferred.

Point of Care and Lateral Flow

The methods are preferably suitable for point of care applications. Such point of care applications, which allow clinical results to be obtained outside of a clinical laboratory setting (e.g. in the Doctor's office, an ambulance, the home, the field) share the common feature of being rapid, and therefore allowing rapid treatment of the patient or modulation of the treatment of the patient. A point of care assay or device is therefore defined as an assay or device which does not require a laboratory setting and which preferably gives immediate results (e.g. within an hour, 50, 40, 30, 20, 15, 10 or 5 minutes of initiating the assay, e.g. of applying the sample to the lateral flow assay). Point of care assays and devices are preferably portable assays and/or devices that can be used by non-specialists (e.g. non-healthcare professionals).

Lateral flow assays (LFAs) are examples of point of care devices. Recent technology has allowed immunoassays to be miniaturized and compartmentalized so as to be usable as test strips by non-healthcare professionals. In certain embodiments therefore the immunoassay is performed in lateral flow assay format. Lateral flow assays are thus a form of immunoassay in which the test sample flows along a test strip. Lateral flow assays (LFAs) are commonly used for point of care assays in view of their relatively low cost and simple operation, and lack of requirement for specialized equipment. Lateral flow assays in general are in the form of single use, disposable test kits.

A sample is in general applied to the test strip and the force of capillary action draws a solvent, in a lateral fashion, through capillary beds formed in or on a substrate through a series of active regions on the test strip to provide a complete immunoassay reaction and provide a recognizable result at the other end of the test strip by the time the solvent/reaction has reached the end of the strip.

The readout is in the form of one or more signals which can be detected. The signals are observed visually or read by an appropriate reader. Whilst historically LFAs were singleplex and read by eye, therefore having limited sensitivity, multiplex and quantitative LFAs are now available. For example desktop and handheld readers for LFAs are now commonplace, and new detection molecules are increasing the options for multiplexing. Appropriate software may be also used to enable a tablet or smartphone to function as a LFA reader, and/or a LFA reader may interpret results that have been obtained by photographing a LFA, e.g. using a tablet or smartphone. Devices that incorporate a tablet or smartphone may also be used as LFA readers.

The typical LFA test strip includes a series of regions that provide various components of the immunoassay. Generally, the first element or portion includes a sample pad, to which the sample is applied. This acts as a sponge and holds an excess of sample fluid. Its function is to transport the sample to the other components of the LFA. In some embodiments the sample pad will pretreat the sample before its transportation (e.g. to separate sample components, to remove interferences, to adjust pH, etc.). An example of such a pretreatment is the use of a filter element in, on, or adjacent to the sample pad. This may e.g. filter particulates from the sample, such as to remove or retard blood cells from whole blood. This may enable plasma to further travel through the device. Suitable filters for removing or retarding cellular material present in blood are well known in the art.

In some embodiments, the fluid migrates to a release pad. This contains absorbed but not immobilised reagents that are needed to carry out the immunoassay (e.g. antibody or modified version of the analyte(s)). As the solvent front moves along the flow path, it dissolves these reagents and brings them into contact with the analyte that is present in the sample. In other embodiments the reagents needed to carry out the immunoassay can be mixed with the sample before applying this mixture to the LFA. In yet further embodiments some reagents are adsorbed on a release pad and others are mixed with the sample before applying the mixture to the LFA.

A typical LFA contains a membrane which is a porous material that provides a path for the flow of material released from the sample and release pads (e.g. a nitrocellulose, cellulose acetate or glass fibre membrane) and also comprises immobilized reagent or reagents (e.g. antibody and/or modified version of the analyte) that participate in the detection of the analyte. The presence or absence of signal at the location of the immobilized reagent or reagents and/or the intensity of the signal at this location gives the readout of the assay. The presence and/or quantity of any analyte is determined by detecting the signal at this location. For example in a sandwich assay, the antibody to the analyte is immobilised at this location and the presence of signal indicates that the “antibody-analyte-antibody” sandwich has been formed. Likewise, in a competitive assay either antigen or modified analyte is immobilised here and the presence or quantity of analyte is detected by detecting a reduction in or absence of signal.

This membrane may optionally include one or more control binding area. This control binding area is co-located within the membrane test strip, and is designed to react with a test sample fluid to form at least one detectable and measurable band, indicating the validity and the completion of the analyte assay test. By way of example, a control binding area may be used to verify that the sample flow is as expected. The control binding area is preferably a spatially distinct region at which a signal may be generated that is indicative of the proper flow of reagents. The control binding area may, for example, contain an analyte of interest, or a fragment thereof, to which excess labeled antibody used in the analyte assay can bind. In operation, a labeled reagent binds to the control zone, even when the analyte of interest is absent from the test sample. The use of a control is helpful in that appearance of a signal indicates the time at which the test result can be read, even for a negative result. Thus, when the expected signal appears in the control, the presence or absence of a signal for the test analyte can be noted. The device may further comprise a negative control area. The purpose of this control area is to alert the user that the test device is not working properly. When working properly, no signal or mark should be visible in the negative control area.

An adsorbent pad may also be present, which works as sink at the end of the strip. It also helps in maintaining flow rate of the liquid over the membrane and stops back flow of the sample.

The components of the LFA are arranged to ensure contact and continuity between the components and to ensure continuity for the capillary flow path. This can be achieved, for example, by providing an overlap between the components of at least 0.5, 1, 2, or 3 mm.

One or more, but preferably all of the components may be disposed on a backing, to provide additional mechanical strength and stability. The backing may be rigid (such as a glass or ceramic or metal backing) or flexible (such as card or plastic).

The assay may be disposed within a housing. The housing provides further mechanical stability, protects the various components, and improves convenience of the device (e.g., providing hand-holds, increases shelf-life, etc.). The housing may be plastic or any other convenient material. The housing may have windows for loading sample and reading test results, etc.

In some embodiments the LFA is an assay in which creatinine and ciclosporin are each detected or quantified using a competitive assay. In some embodiments the LFA is an assay in which creatinine and ciclosporin are each detected or quantified using a sandwich assay. In some embodiments the LFA is an assay in which creatinine is detected or quantified using a sandwich assay and ciclosporin is detected or quantified using a competitive assay. In some embodiments the LFA is an assay in which creatinine is detected or quantified using a competitive assay and ciclosporin is detected or quantified using a sandwich assay.

In some embodiments the LFA is an assay in which creatinine and tacrolimus are each detected or quantified using a competitive assay. In some embodiments the LFA is an assay in which creatinine and tacrolimus are each detected or quantified using a sandwich assay. In some embodiments the LFA is an assay in which creatinine is detected or quantified using a sandwich assay and tacrolimus is detected or quantified using a competitive assay. In some embodiments the LFA is an assay in which creatinine is detected or quantified using a competitive assay and tacrolimus is detected or quantified using a sandwich assay.

Appropriate configurations of reagents can be devised for each of these alternatives, e.g. using one or more of antibodies that specifically bind to tacrolimus, antibodies that specifically bind to creatinine, antibodies that specifically bind to ciclosporin, modified tacrolimus, modified creatinine, modified ciclosporin.

Detecting and Quantifying the Signal

Various labels can be used to obtain the read-out in a LFA. Any of the labels mentioned above for immunoassays may be employed. Appropriate detection systems are known for each type of label and some examples of preferred labels are provided below.

The most common label or reporter entity used in LFAs is colloidal gold. Reagents can be noncovalently or covalently bound or attached to gold, and visual detection of the signal can be simple and robust. Gold is stable under exposure to heat and light; degradation is limited primarily by the stability of the protein(s). These particles have very high affinity toward biomolecules and can be easily functionalized. Their unique features include environment friendly preparation, high affinity toward proteins and biomolecules, enhanced stability, exceptionally higher values for charge transfer and good optical signaling.

Fluorescent labels can also be used. An advantage of fluorescence over absorbance systems is the dark and uniform background that is achieved by efficient blocking of the excitation light. Fluorescence detection also provides a wide dynamic range since the light emitted is proportional to the concentration while the amount of light reflected after absorption is a nonlinear function of concentration. Generally, fluorescence systems tend to be more expensive due to the expensive light sources required to illuminate the fluorescent reporters, the interference filters and detection systems required to process and capture the emitted light, and the data processing required to produce the result. High photostability and brightness are in general required for LFAs.

Quantum dots display very unique electrical and optical properties. These semiconducting particles are not only water soluble but can also be easily combined with biomolecules because of closeness in dimensions, and are an alternative to organic fluorescent dyes. Like gold nanoparticles QDs show size dependent optical properties and a broad spectrum of wavelengths can be monitored. Single light source is sufficient to excite quantum dots of all different sizes. QDs have high photo stability and absorption coefficients.

Upconverting phosphors (UCP) are characterized by their excitation in infra-red region and emission in high energy visible region. Compared to other fluorescent materials, they do not showing auto fluorescence and they do not photo degrade biomolecules.

Other fluorescent labels used in LFA include silica nanoparticles, and fluorescent microspheres.

Magnetic labeled particles can also be used. Like the fluorescent particles these require the use of an electronic reader to assess the test result. Coloured magnetic particles produce colour at the test line which is measured by an optical strip reader but magnetic signals coming from magnetic particles can also be used as detection signals and recorded by a magnetic assay reader.

Enzymes are also employed as labels in LFA (e.g. horseradish peroxidase labeled antibody conjugates). Where enzymes are used, sensitivity of detection is dependent on the enzyme-substrate combination that is chosen.

Colloidal carbon has been used as a label in LFA and carbon black nanoparticles show very low detection limits compared to other labels.

The detection system that is used will depend on the nature of the label. In case of colour producing labels, qualitative or semi-quantitative analysis can be done by visual inspection of colours at test and control lines. For quantification, optical strip readers can be employed for measurement of the intensity of colours produced at test and control lines of strip. Such strip readers record the intensity of the signal using imaging software. Alternatively, optical images of the strips can also be recorded (e.g. with a camera and then processed using appropriate software). For other labels, appropriate test readers are also known in the art. Selection of the detector is mainly determined by the label employed in analysis.

To facilitate quantitation, standards containing known amounts of analyte can be applied to the LFA. By comparing the signal that is achieved with the signal generated from one or more known amount of analyte the amount of the analyte in the sample may be quantitated. These steps can be carried out as part of the method or may be incorporated into any automatic reading device that is used.

Method Steps

Where the method is based on a LFA, the method may include the step of loading the LFA (e.g. the test strip) with the sample (e.g. at the sample pad), and incubating the LFA under conditions whereby the analyte(s) of the sample and any reagents that are released from the conjugate pad or that are added to the sample migrate by capillary flow through the membrane and the analyte(s) binds to an antibody that is released from the conjugate pad, added to the sample, or that is present on the membrane of the lateral flow assay, and detecting and/or quantifying the analyte(s). Detection and/or quantifying the analyte(s) is carried out as described elsewhere herein.

Determining the Presence or Quantity of Analyte

As used herein, a “method for detecting” includes a method in which it is determined whether the relevant molecule is present, irrespective of whether the molecule is actually found, and therefore can alternatively be defined as a method of determining the whether the molecule is present or absent. Methods for detecting the molecules thus include methods in which the recited steps are carried out but which it is not determined that the molecule is present.

A method of quantifying involves determining the amount or concentration of the molecule that is present in the sample. This can be achieved by various methods. For example in the context of a lateral flow assay, it is possible to measure the intensity of a signal to determine the quantity of analyte in the sample. In general this will be carried out by a reader (e.g. a lateral flow reader) which may be a handheld device. For example, by utilizing unique wavelengths of light for illumination in conjunction with either CMOS or CCD detection technology, a signal rich image can be produced of the actual test lines. Using image processing algorithms specifically designed for a particular test type and medium, line intensities can then be correlated with analyte concentrations. An exemplary handheld lateral flow device platform is made by Detekt Biomedical L.L.C. Alternatively, appropriate software can be incorporated into a smartphone or tablet to enable this to become a handheld lateral flow reader.

Alternative non-optical techniques are also able to report quantitative assays results. One such example is a magnetic immunoassay (MIA) in the lateral flow test form also allows for getting a quantified result. Alternatively, the configuration of the lateral flow assay may itself give rise to quantitation (see e.g. [5]).

Detecting in a Single Assay

Prior art methods, such as those described in US20090298106, exist whereby a single small volume blood sample is taken, but this is split into different subsamples, with one subsample being analysed using LC-MS/MS for the presence of immunosuppressive drugs and other subsamples being analysed using standard chemical assays for the presence of kidney or hepatic function makers. In contrast, the present method preferably detects or quantifies creatinine and the immunosuppressive drug in one assay, e.g. using a single LFA.

Preferably the one assay involves the simultaneous detection or quantification of creatinine and the immunosuppressive drug, e.g. in a single assay (e.g. using a single sample, e.g. applied at a single location on a LFA). In the context of an immunoassay this may be achieved by the presence of immobilised reagents for the detection of each of the analytes (e.g. on a single lateral flow strip and/or a single lateral flow device). In the context of a lateral flow assay the strip (e.g. single strip) may contain immobilised reagents for the detection of each of the analytes. These may be presented in any appropriate format. In a preferred embodiment a plurality of such locations are present, each corresponding to a different analyte and each comprising reagents that allow the detection of the appropriate analyte, can be provided on a single solid support. These locations are preferably non-contiguous so that a border that is not part of either location completely surrounds each of the areas. This enables the detection of a signal that is specific to each antibody/analyte. This in turn allows the detection and/or quantification of more than one analyte in one assay e.g. using a single LFA.

The plurality of locations may be presented e.g. in series, in parallel, in combination thereof, and/or as an array. If the locations are presented in series, this means that the solvent front crosses the reagent zones containing immobilised reagents to detect one of the analytes and subsequently crosses one or more other reagent zone containing immobilised reagents to detect one or more other analyte. If the locations are presented in parallel, the reagent zones containing immobilised reagents to detect the analytes are arranged in different (e.g. parallel) lanes. In some embodiments the device is configured so as to allow sample application at a single location. A single assay thus allows simultaneous detection or quantification of the two analytes, from a single sample, and a single assay thus contains the reagents (or all of the reagents) required to generate one or more readout or signal that in turn allows the detection or quantification of each of the two analytes.

Lateral flow strips for this purpose can be built in various ways i.e. by increasing the length of the test strip and/or increasing the number of test lines on conventional strip. Modifying the structure of the test strip is also possible (see e.g. [6]). Any configuration may be used in such a lateral flow assay test strip.

Additionally or alternatively the detection and/or quantification of more than one analyte in one assay e.g. using a single LFA can be achieved by using more than one label (e.g. a plurality of labels).

Sample

The sample is a blood sample. It may be whole blood, or a blood component, such as serum or plasma, may be used. The sample is preferably a liquid sample.

The sample is preferably a small sample, such as a sample obtained from a fingerprick or earlobe prick. Such samples can be obtained by the patient. It can therefore be a small volume. Exemplary volumes for the sample are less than 500 μl, 400 μl, 300 μl, 200 μl, 150 μl, or 100 μl.

The sample may be used directly in the assay or may be subject to treatment, e.g. diluted for use in the assay (e.g. in an aqueous solution, such as a buffer, or chelator, such as EDTA), or treated e.g. to lyse blood components such as blood cells, e.g. red and/or white blood cells. Lysing blood cells may be advantageous to e.g. release tacrolimus. If the sample is diluted prior to use in the assay, it preferably contains less than 99, 98, 95, 90, 85, 80, 75, or 50% diluent. The aqueous solution may contain one or more reagents of an immunoassay.

Methods according to the present invention may therefore additionally comprise one or more of isolating plasma or serum or other blood components from blood; mixing the sample or fraction thereof with an aqueous solution, such as a buffered aqueous solution, which may optionally contain one or more reagents of an immunoassay. Lysing blood cells may form an optional additional step.

The methods of the invention may further comprise the step of obtaining the blood sample from the patient.

Patient

The sample is from a patient (e.g. a patient who has received an organ or tissue transplant) and who is undergoing or who has undergone treatment with an immunosuppressive drug selected from ciclosporin and tacrolimus. A patient who has undergone treatment with the immunosuppressive drug has preferably been administered with the drug within a period of 1, 2, 3, 4, 5 or 6 weeks preceding the date at which the sample was taken. A patient has preferably been administered the immunosuppressive drug for a period of at least 1, 2, 3, 4, 5 or 6 weeks. Administration of the immunosuppressive drug may have been by any conventional means, e.g. orally, intravenously.

The patient may have received a transplant of any organ or tissue, but the organ or tissue is preferably an organ selected from kidney, heart, liver, intestine (e.g. small intestine), thymus, pancreas, lung and trachea or a tissue selected from skin, bone, bone marrow, tendon, heart valve, cornea, nerve or vein.

The patient may be a human or non-human organism, preferably a human, although the methods are applicable to both human and veterinary patients.

The patient may have or may be at risk of developing reduced renal function and/or nephrotoxicity. Nephrotoxicity is a term used to describe a poisonous effect of some substances, including immunosuppressive drugs, on the kidneys.

The term “renal function” is used to describe the state of health of the patient's kidneys, including their excretory function, as determined by a test or assay described herein or as is well known in the art. The renal function may be monitored by determining the level of creatinine in the sample using methods as defined herein. If the renal function is normal (e.g. good), the blood creatinine levels would fall within about 0.6 and about 1.2 mg/dL or about 53 to about 106 μmol/L for men, within about 0.5 and about 1.1 mg/dL or about 44 to about 97 μmol/L for women, within about 0.5 and about 1.0 mg/dL for teens, within about 0.3 and about 0.7 mg/dL for children, and within about 0.3 and about 1.2 mg/dL for newborns. Creatinine levels above these ranges may be indicative of reduced renal function and/or nephrotoxicity.

Device/Equipment

The invention further provides a device for carrying out the method of the invention. The device is therefore preferably a lateral flow device suitable for carrying out lateral flow assay, as described herein. Preferably the device is suitable for the detection of creatinine and an immunosuppressive drug selected from tacrolimus and ciclosporin in a blood sample. The device may be adapted or configured to allow any of the features described above.

Preferred assay devices of the present invention will comprise a lateral flow assay device configured to perform a sandwich and/or competitive immunoassay for one or more of the analytes referred to above (e.g. tacrolimus and creatinine or ciclosporin and creatinine).

Preferably the device is configured for multiplex detection so that creatinine and an immunosuppressive drug selected from tacrolimus and ciclosporin are detected and/or quantified using a single sample, and preferably using a single LFA.

Appropriate reagents may be present on any LFA device and/or may be present in reagents that are mixed with the sample prior to applying the sample to the LFA device.

Preferably the device is configured to be read with a handheld reader as discussed elsewhere herein, e.g. for quantifying the analyte. The device may further comprise a handheld reader.

In various related aspects, the present invention relates to kits for performing the methods described herein. Suitable kits comprise (i) devices and/or reagents sufficient for performing at least one of the described methods, and (ii) instructions for performing the methods.

Methods for producing the device are also provided. Such methods involve applying reagents to the conjugate release pad (e.g. in the form of an aqueous solution, in which case the applying can include drying the conjugate release pad under conditions to adsorb but not immobilize the conjugate), and/or immobilising reagents to the membrane. The method may further comprise assembling the test strip by overlapping the sample pad, release pad, membrane, and absorbent pad as described herein. Such assembly may be carried out on the backing when present. The assembled test strip may be placed within the housing when present.

Methods of Treatment or Monitoring

The invention further provides a method that enables the reduction of nephrotoxicity in a patient (e.g. an organ or tissue transplant patient) who is undergoing treatment with an immunosuppressive drug selected from tacrolimus and ciclosporin at a first dose, the method comprising quantifying creatinine and the immunosuppressive drug in a blood sample from the patient, and if the level of creatinine is indicative of nephrotoxicity, determining a new dose of the immunosuppressive drug for the patient.

The invention further provides a method that enables the maintenance of good renal function in a patient (e.g. an organ or tissue transplant patient) who is undergoing treatment with an immunosuppressive drug selected from tacrolimus and ciclosporin at a first dose, the method comprising quantifying creatinine and the immunosuppressive drug in a blood sample from the patient, and if the level of creatinine is indicative of reduced renal function, determining a new dose of the immunosuppressive drug for the patient. This can alternatively be defined as enabling a reduction in the occurrence or length of time during which a patient has reduced renal function.

Any reduced renal function can be a result of nephrotoxicity (in which case the dose of the immunosuppressive drug should be reduced) or, in the case of a kidney transplant patient, it can be the result of kidney graft rejection (in which case the dose of the immunosuppressive drug may be maintained or increased).

The methods may further comprise the step of administering the immunosuppressive drug at the same dose or at a new dose. The new dose is preferably about, or less than 120, 115, 110, 105, 100, 95, 90, 80, 70, 60, or 50% of the first dose. When the dose is reduced to reduce nephrotoxicity, this can be described as a method of reducing nephrotoxicity in the patient. When the dose is reduced to maintain good renal function in a patient, this can be described as a method of maintaining good renal function in a patient, or a method of reducing the occurrence or length of time during which a patient has reduced renal function. When the dose is maintained or increased to maintain good renal function in a patient, this can be described as a method of maintaining good renal function in a patient, or a method of reducing the occurrence or length of time during which a patient has reduced renal function.

The method can also be defined as a method for determining nephrotoxicity and immunosuppressive drug levels in a patient (e.g. an organ or tissue transplant patient) who is undergoing treatment with an immunosuppressive drug selected from tacrolimus and ciclosporin, the method comprising quantifying creatinine and the immunosuppressive drug in a blood sample from the patient.

The method can also be defined as a method for determining renal function and immunosuppressive drug levels in a patient (e.g. an organ or tissue transplant patient) who is undergoing treatment with an immunosuppressive drug selected from tacrolimus and ciclosporin, the method comprising quantifying creatinine and the immunosuppressive drug in a blood sample from the patient.

The method can also be defined as a method for monitoring nephrotoxicity and immunosuppressive drug levels in a patient (e.g. an organ or tissue transplant patient) who is undergoing treatment with an immunosuppressive drug selected from tacrolimus and ciclosporin, the method comprising quantifying creatinine and the immunosuppressive drug in a blood sample from the patient.

The method can also be defined as a method for monitoring renal function and immunosuppressive drug levels in a patient (e.g. an organ or tissue transplant patient) who is undergoing treatment with an immunosuppressive drug selected from tacrolimus and ciclosporin, the method comprising quantifying creatinine and the immunosuppressive drug in a blood sample from the patient.

Any of the methods and devices described herein may be used.

Alternatively stated the invention provides the use of the methods, devices and kits in monitoring a patient (e.g. an organ or tissue transplant patient). For example the monitoring may be of nephrotoxicity and immunosuppressive drug levels in an organ or tissue transplant patient who is undergoing treatment with an immunosuppressive drug selected from tacrolimus and ciclosporin, or of renal function and immunosuppressive drug levels in an organ or tissue transplant patient who is undergoing treatment with an immunosuppressive drug selected from tacrolimus and ciclosporin.

“Monitoring” includes for example carrying out the method or the uses of the devices or the kits multiple times (e.g. at least 2, 3, 4, 5, 6, 7, 8, 9 or 10) over a period of time (e.g. over a period of at least 1, 2, 3, 4 weeks or 1, 2, 3, 4, 5, 6, 7, 8, 9, 10, 11, 12 months or 1, 2, 3, 4, 5 years).

General

The term “analyte” is meant to refer to a component of a sample that is to be detected or quantified and includes creatinine, tacrolimus and ciclosporin.

The term “comprising” encompasses “including” as well as “consisting” e.g. a composition “comprising” X may consist exclusively of X or may include something additional e.g. X+Y.

The term “about” in relation to a numerical value x means, for example, x±10%.

It will be understood that the invention has been described by way of example only and modifications may be made whilst remaining within the scope and spirit of the invention.

The invention may be described by the following numbered embodiments:
1. A method of detecting or quantifying creatinine and an immunosuppressive drug selected from tacrolimus and ciclosporin in a blood sample.
2. The method of embodiment 1, wherein creatinine and the immunosuppressive drug are detected or quantified together in a single assay.
3. The method of any preceding embodiment, wherein creatinine and the immunosuppressive drug are detected or quantified using immunoassays.
4. The method of any preceding embodiment, wherein at least one immunoassay is a competitive immunoassay.
5. The method of any one of embodiments 3 to 4, wherein the immunoassays are present in a lateral flow assay format.
6. The method of any preceding embodiment which is a method of quantifying creatinine and an immunosuppressive drug selected from tacrolimus and ciclosporin in a blood sample.
7. The method of any preceding embodiment, wherein the quantification is carried out using a scan reader.
8. A lateral flow device for carrying out the method of any one of embodiments 1 to 6.
9. The device of embodiment 8 further comprising a LFA reader, which preferably is or comprises a smartphone or tablet, for carrying out quantification.
10. A kit comprising the device of embodiment 8 or 9 and instructions for performing the methods.
11. A method that enables the reduction of nephrotoxicity in an organ or tissue transplant patient who is undergoing treatment with an immunosuppressive drug selected from tacrolimus and ciclosporin at a first dose, the method comprising quantifying creatinine and the immunosuppressive drug in a blood sample from the patient, and if the level of creatinine is indicative of nephrotoxicity, determining a new dose of the immunosuppressive drug for the patient.
12. A method of that enables the maintenance of good renal function in an organ or tissue transplant patient who is undergoing treatment with an immunosuppressive drug selected from tacrolimus and ciclosporin at a first dose, the method comprising quantifying creatinine and the immunosuppressive drug in a blood sample from the patient, and if the level of creatinine is indicative of reduced renal function, determining a new dose of the immunosuppressive drug for the patient.
13. The method of embodiment 11 or 12, further comprise the step of administering the immunosuppressive drug at the new dose.
14. The method of embodiment 13 wherein the new dose is about, or less than 120, 115, 110, 105, 100, 95, 90, 80, 70, 60, or 50% of the first dose.
15. A method for determining nephrotoxicity and immunosuppressive drug levels in an organ or tissue transplant patient who is undergoing treatment with an immunosuppressive drug selected from tacrolimus and ciclosporin, the method comprising quantifying creatinine and the immunosuppressive drug in a blood sample from the patient.
16. A method for determining renal function and immunosuppressive drug levels in an organ or tissue transplant patient who is undergoing treatment with an immunosuppressive drug selected from tacrolimus and ciclosporin, the method comprising quantifying creatinine and the immunosuppressive drug in a blood sample from the patient.
17. A method for monitoring nephrotoxicity and immunosuppressive drug levels in an organ or tissue transplant patient who is undergoing treatment with an immunosuppressive drug selected from tacrolimus and ciclosporin, the method comprising quantifying creatinine and the immunosuppressive drug in a blood sample from the patient.
18. A method for monitoring renal function and immunosuppressive drug levels in an organ or tissue transplant patient who is undergoing treatment with an immunosuppressive drug selected from tacrolimus and ciclosporin, the method comprising quantifying creatinine and the immunosuppressive drug in a blood sample from the patient.
19. The method of any one of embodiments 11 to 18, having features as defined in any of embodiments 2 to 7.
20. Use of the method of any one of embodiments 1 to 7, 11, 12, 15 to 19 or the device of any one of embodiment 8 to 9 or the kit of embodiment 10 in monitoring an organ or tissue transplant patient.
1 Shipkova, M et al., Clinical Biochemistry 47 (2014) 1069-1077
2 http://bestpractice.bmj.com/best-practice/monograph/935.html
3 van Erp et al, J. Immunoassay 12: 425-43, 1991
4 WO2014070686
5 WO2013140089
6 US20080317633

1. A method of detecting or quantifying creatinine and an immunosuppressive drug selected from tacrolimus and ciclosporin in a blood sample, wherein the creatinine and the immunosuppressive drug are detected or quantified using immunoassays that are present in a lateral flow assay format.

2. The method of claim 1, wherein the creatinine and the immunosuppressive drug are detected or quantified together in a single assay.

3. The method of claim 1, wherein at least one immunoassay is a competitive immunoassay.

4. The method of claim 1, the method comprising quantifying the creatinine and the immunosuppressive drug selected from tacrolimus and ciclosporin in the blood sample.

5. The method of claim 1, wherein the quantifying is carried out using a scan reader.

6. A lateral flow device for carrying out the method of claim 1.

7. The device of claim 6 further comprising a LFA reader for carrying out quantification.

8. A kit comprising the device of claim 6 and instructions for using the device.

9. A method that enables the reduction of nephrotoxicity in an organ or tissue transplant patient who is undergoing treatment with an immunosuppressive drug selected from tacrolimus and ciclosporin at a first dose, the method comprising quantifying creatinine and the immunosuppressive drug in a blood sample from the patient using the method of claim 1, and if the level of creatinine is indicative of nephrotoxicity, determining a new dose of the immunosuppressive drug for the patient.

10. A method of that enables the maintenance of good renal function in an organ or tissue transplant patient who is undergoing treatment with an immunosuppressive drug selected from tacrolimus and ciclosporin at a first dose, the method comprising quantifying creatinine and the immunosuppressive drug in a blood sample from the patient using the method of claim 1, and if the level of creatinine is indicative of reduced renal function, determining a new dose of the immunosuppressive drug for the patient.

11. The method of claim 9, further comprising the step of administering the immunosuppressive drug at the new dose.

12. The method of claim 11 wherein the new dose is about, or less than 120, 115, 110, 105, 100, 95, 90, 80, 70, 60, or 50% of the first dose.

13. A method for determining nephrotoxicity and immunosuppressive drug levels in an organ or tissue transplant patient who is undergoing treatment with an immunosuppressive drug selected from tacrolimus and ciclosporin, the method comprising quantifying creatinine and the immunosuppressive drug in a blood sample from the patient using the method of claim 1.

14. A method for determining renal function and immunosuppressive drug levels in an organ or tissue transplant patient who is undergoing treatment with an immunosuppressive drug selected from tacrolimus and ciclosporin, the method comprising quantifying creatinine and the immunosuppressive drug in a blood sample from the patient using the method of claim 1.

15. A method for monitoring nephrotoxicity and immunosuppressive drug levels in an organ or tissue transplant patient who is undergoing treatment with an immunosuppressive drug selected from tacrolimus and ciclosporin, the method comprising quantifying creatinine and the immunosuppressive drug in a blood sample from the patient using the method of claim 1.

16. A method for monitoring renal function and immunosuppressive drug levels in an organ or tissue transplant patient who is undergoing treatment with an immunosuppressive drug selected from tacrolimus and ciclosporin, the method comprising quantifying creatinine and the immunosuppressive drug in a blood sample from the patient using the method of claim 1.

17. The method of claim 10 further comprising the step of administering the immunosuppressive drug at the new dose.

18. The method of claim 17, wherein the new dose is about, or less than 120, 115, 110, 105, 100, 95, 90, 80, 70, 60, or 50% of the first dose.

19. The device of claim 7, wherein the LFA reader comprises a smartphone or tablet.
